(19) United States
(12) Patent Application Publication
(10) Pub. No.: US 2024/0371497 A1
(43) Pub. Date: Nov. 7, 2024

(54) SYSTEM AND METHOD FOR DIAGNOSTICS AND FINDING TREATMENTS FOR MILD COGNITIVE IMPAIRMENT, DEMENTIAS AND NEURODEGENERATIVE DISEASES

(71) Applicant: United Arab Emirates University, Al Ain (AE)

(72) Inventors: Yauhen STATSENKO, Al Ain (AE); Tetiana HABUZA, Al Ain (AE); Taleb AL MANSOORI, Al Ain (AE); Klaus Neidl-Van GORKOM, Al Ain (AE); Juri G. GELOVANI, Al Ain (AE); Milos LJUBISABLJEVIC, Al Ain (AE)

(21) Appl. No.: 18/766,050

(22) Filed: Jul. 8, 2024

Related U.S. Application Data
(63) Continuation-in-part of application No. 18/143,128, filed on May 4, 2023, now abandoned.

(51) Int. Cl. G16H 20/70; A61B 5/00; G16H 50/20; G16H 50/70

(52) U.S. Cl. CPC G16H 20/70; A61B 5/4064; A61B 5/4088; G16H 50/20; G16H 50/70

(57) ABSTRACT

The invention relates to a system and a method for finding treatments for mild cognitive impairment, dementias and neurodegenerative diseases, the method including collecting brain imaging, laboratory, functional data from a group of cognitively normal individuals, patients with confirmed cases of at least one of the diagnoses, and an examinee with unknown diagnosis; entering the diagnostic data obtained in the previous steps into a computing device; using a machine learning system to produce a plurality of cross-modal regression models specific for each diagnosis; producing the classification module that identifies a disease-specific cross-modal regression model which optimally fits an individual case; assembling an ensemble model from the disease-specific cross-modal regression models and the classification module; deploying and running the ensemble model at the computing device to calculate probabilities of the diseases, output a diagnosis with the highest probability and select the optimal therapeutic plan comprising at least one treatment option.

### CROSS-REFERENCE TO RELATED APPLICATIONS

This application is a continuation of the application for Letters Patent of the United States of America describing the same and based thereon (U.S. patent application Ser. No. 18/143,128) by Yauhen STATSENKO, Tetiana HABUZA, Taleb AL MANSOORI, Klaus Neidl-Van GORKOM, Juri G. GELOVANI, and Milos LJUBISABLJEVIC having invented certain inventions and improvements in SYSTEM AND METHOD FOR DIAGNOSTICS OF MILD COGNITIVE IMPAIRMENT, DEMENTIAS AND NEURODEGENERATIVE DISEASES, and having filed an application. This patent application is herein incorporated by reference in its entirety.

### STATEMENT REGARDING PRIOR DISCLOSURES BY AN INVENTOR OR JOINT INVENTOR

Some aspects of the invention were disclosed in the following documents:
1) Statsenko, Yauhen, et al. “Patterns of structure-function association in normal aging and in Alzheimer's disease: Screening for mild cognitive impairment and dementia with ML regression and classification models.” Frontiers in Aging Neuroscience 14 (2023): 943566. DOI: 10.3389/fnagi.2022.943566
2) Habuza, T. (2022). DIAGNOSTICS OF DEMENTIA FROM STRUCTURAL AND FUNCTIONAL MARKERS OF BRAIN ATROPHY WITH MACHINE LEARNING. Electronic Theses and Dissertation. Defended on June 2022 available at https://scholarworks.uaeu.ac.ae/all_dissertations/161/on 25 May 2024

### BACKGROUND OF THE INVENTION

Computer-aided decision systems have been proposed in US2008/0101665A1 COLLINS and US2023/0225668 A1 TSOKOS. COLLINS uses MRI data obtained from an individual to predict the progression of his mental status, whereas TSOKOS relates to predication of progression of Alzheimer disease. Thus, the known systems do only predict an outcome of a single known illness, i.e. Alzheimer's Disease. An object of the present invention is to overcome the disadvantages of the prior art. The existing methods for multimodal diagnostics have many disadvantages including poor accuracy which delays disease identification and treatment: these methods may detect dementia when the cognitive resources are exhausted, and the cognition-focused interventions do not help the patients.

The drawbacks of the conventional methods of treating cognitive-focused treatment are multifold: they are expensive, time—and recourse-consuming: the medical staff, rehabilitative devices and techniques required for the procedures are limited. Therefore, the conventional methods of treating cognitive-focused treatment are prescribed for indications after the diagnosis is established. The therapeutic potential of disease-modifying or palliative treatment is considerable in early disease stages. As there are many impairments that could lead to mild cognitive problems, it is difficult to select a suitable treatment and often more exotic illnesses are not diagnosed correctly as normally only near-fetched illnesses are taken into consideration.

In a combined examination, the subject is scanned in multiple diagnostic modes, and the multimodal diagnostics should benefit from the advantages of various methods and overcome their disadvantages. Conventional methods for establishing the diagnosis and detecting indications for cognition-focused interventions may combine data analysis of distinct diagnostic modalities, but they do not comprise disease-specific cross-modal regression models. The systems suggested in the prior art are incapable of incorporating an infinite number of models to identify a suspected disease among many diseases.

To overcome the aforementioned drawbacks, there is a need to provide a system and a method for a new holistic approach to disease management that is an enhancement to the conventional systems and methods for diagnostics and finding treatments for mild cognitive impairment, dementias and neurodegenerative diseases.

### BRIEF SUMMARY OF THE INVENTION

This problem is solved by a system according to claim 2 and a method for diagnostics and finding treatments for mild cognitive impairment, dementias and neurodegenerative diseases according to claim 1. Developments thereof are the subject of the claims dependent thereon.

The method according to the invention incorporates a computing system to detect the best-fitting model from a plurality of disease-specific cross-modal regression models trained with real data and thus identify early indications for effective treatment of a suspected disease according to a plurality of diagnostic data obtained from the individual. The desired system is capable of incorporating an infinite number of disease-specific cross-modal regression models. Thus, the invention relates to analysis of an impaired mental status as well as to the search of suitable treatments thereof. There exist also restrictions with regard to the availability and/or costs of possible treatments-therefore it is useful if several treatments are suggested by the system.

In contrast to COLLINS and TSOKOS, the system classifies examinees by diagnoses and helps to identify the optimal treatment. Besides, the methods by COLLINS and TSOKOS forecast changes in the examinee's cognitive status to describe the future disease course. However, the system according to the invention computes cognitive scores on the day of the examination to detect the present cognitive status which may change in the future. The prognostic systems by COLLINS and TSOKOS detect reversible, stable or progressive disease forms but do not give an actual diagnosis.

The concepts of the systems are reflected in the architectures of their models which serve as a numerical description of the reality. The mathematical representation of reality has predictors that are used to compute the targeted variables with a set of equations. Although in COLLINS and TSOKOS imaging data can be used as predictors, the systems differ significantly from the present invention. In COLLINS, the targeted variable is the MCI-to-AD conversion time, whereas in the system according to the invention, the targeted variables are the cognitive score values.

Another difference is the datasets used for training the models. In the system and method according to the invention, data from different populations of patients are used, whereas COLLINS and TSOKOS lack disease-specific cross-modal regression models. In the COLLINS' system, a regression model was trained on findings for decliners, improvers and patients with stable clinical state. The TSOKOS' system does not contain findings on cognitively normal population.

To serve as a physical representation of reality, mathematical models could be trained on various datasets. Hence, the models can reflect some aspects specific to the cohort on which data they were trained.

Another difference between the COLLINS, TSOKOS systems and the present invention system is that the latter can incorporate an infinite number of datasets specific for a disease or health status. The invention provides the system and method for diagnostics and finding treatments for mild cognitive impairment, dementias and neurodegenerative diseases that focus on the change in at least any two types of diagnostics data (brain imaging, functional and laboratory data) in normal and accelerated aging.

The system and method according to the invention are non-invasive, reliable, economically affordable, and they can be used remotely, for example, in the form of telemedicine. Another advantage of the present invention is that it is able to incorporate new scientific data by adding results in genetic and epigenetic tests, new treatment options and new training data for creating new diagnostic models. The invention works as an open system where old data can be combined with new data types and new diagnostics modalities. Due to these advantages, the present invention can be adjusted to new diagnostic methods, data and data types by incorporating new findings. The invention can use standard cognitive tests and routine structural magnetic resonance imaging which are economically affordable.

The advantages of the invention are multifold. First, the present invention may serve as a screening tool for mild cognitive impairment, dementias and neurodegenerative diseases. The system and the method can also be used as a tool for differential diagnostics and differential treatment, since the present invention provides a plurality of disease-specific cross-modal regression models each detecting indications for disease-specific treatment. Second, the present invention supports effective treatment of cognitive disorders by providing indications for cognition-focused interventions in the patients with mild cognitive impairment, dementias and neurodegenerative diseases. If ordered at early disease stages, currently existing therapeutic options hold a potential to delay cognitive decline and they are more efficient than at late stages when the diagnosis can be confirmed with the conventional diagnostic methods. Third, the present invention is capable of incorporating an infinite number of disease-specific cross-modal regression models and to implement multimodal diagnostics into routine medical practice. The multimodal diagnostic approach is more advantageous compared to the unimodal one: once combined, different diagnostic techniques have their strengths leveraged and the final accuracy improved. The accurate diagnostics allows to detect early indications for cognition-focused interventions and to prescribe the treatment in time, before the depletion of structural and cognitive resources of the brain.

The invention relates to a system and a method with the steps from A to I:
(A) Collecting brain imaging data with at least one type of imaging modality from a group of cognitively normal individuals and patients with confirmed cases of at least one diagnosis from a plurality of diseases;
(B) Collecting laboratory data with at least one type of laboratory analysis from the aforesaid individuals and patients of step A;
(C) Collecting functional data with at least one functional test from the aforesaid individuals and patients of step A;
(D) Collecting brain imaging data, laboratory data and functional data from an examinee with unknown diagnosis with at least the majority of diagnostic procedures as discussed in steps A, B, and C;
whereas different types of diagnostics data are brain imaging data, laboratory data and functional data, and steps A, B, C and D are performed by medical staff;
(E) Entering the diagnostic data obtained in step D into a computing device
(F) Entering at least two types of diagnostic data obtained in steps A, B and C into a machine learning system which produces cross-modal regression models specific for each diagnosis and gets output values of the models;
(G) Entering the output values of the plurality of disease-specific cross-modal regression models from step F as predictors and the diagnoses of the individuals and patients of step A as targeted variables into the machine learning system which produces a classification module identifying at least one disease-specific cross-modal regression model which optimally fits an individual case;
(H) Assembling an ensemble model from the disease-specific cross-modal regression models of step F and the classification module of step G and deploying the ensemble model H at the computing device of step E;
(I) Running the ensemble model of step H at the computing device of step E to calculate probabilities of the diseases, output at least one diagnosis with the highest probability and select the optimal therapeutic plan implementing at least one efficient treatment option,

whereas the computing device of steps E, H and I may be at least partially different from the machine learning system of steps F and G.

In one aspect of the invention, the method includes a plurality of disease-specific cross-modal regression models, a classification module, and an ensemble model for performing multi-step analysis and a plurality of disease-specific treatment options for cognition-focused interventions. Other embodiments of the invention include corresponding therapeutic devices, drugs, architecture, apparatus, and computer programs recorded on one or more storage devices, each configured to perform the actions of the methods. Computer-aided decision allows for ordering in-time treatment with a plurality of treatment options for patients with mild cognitive impairment, dementias and other neurodegenerative diseases.

In accordance with an embodiment of claim 2, the proposed system incorporates several steps. In the first step, medical staff uses medical equipment to collect brain imaging data from a group of cognitively normal individuals and patients with confirmed cases of mild cognitive impairment, dementias and neurodegenerative diseases. In the second and third steps, laboratory data and functional data are collected from the same groups of individuals and patients. In the fourth step, the medical staff collects brain imaging data, laboratory data and functional data of an examinee to be diagnosed and treated. Further, in the fifth step, the diagnostic data are entered into a computing device. In the sixth step, a machine learning system is used to train a plurality of disease-specific cross-modal regression models predicting one type of diagnostic data (functional or laboratory or brain imaging data) from at least one other type of diagnostic data. In the sevenths step, the machine learning system is trained to produce a classification module detecting the correct clinical diagnosis and the optimal treatment from the output of the disease-specific cross-modal regression models. In the eighths step, an ensemble model is assembled from the disease-specific cross-modal regression models and the classification module. The examiner enters the ensemble model into a computing device and stores a list of treatment options for each diagnosis in the computing device. In the ninths step, the ensemble model is provided with the plurality of diagnostic data received in the fourth step. The ensemble model is run with the functional, laboratory and/or brain imaging findings as input, and calculates probabilities of mild cognitive impairment, dementias and neurodegenerative disease. The output of the ensemble model is at least one diagnosis with the highest probability. The computing device finds at least one treatment option for the diagnosis from the list of available treatment options, thus, providing indications for a cognition-focused intervention: so treatment can be started on basis of the system and method according to the invention.

In accordance with an embodiment of claim 3, brain imaging data is any one or a combination of voxel-based morphometry data, surface-based morphometry data, radiomics findings, brain-imaging data, angiography findings, metabolic imaging data, and blood oxygen level dependent images.

In accordance with an embodiment of claim 4, laboratory data are selected from the group consisting of biochemical, hormonal, immunologic, hematologic analytic data, and any other type of biological data obtained from laboratory tests of human samples and combinations thereof.

In accordance with an embodiment of claim 5, the individual passes one or more functional examinations that are selected from the group comprising cognitive, psychophysiological, neurophysiological tests, any other type of functional examination and assessment and combinations thereof.

In accordance with an embodiment of claim 6, the disease-specific cross-modal regression models are trained to predict diagnostic data of one type from the diagnostic data of at least one other type, and each model of the plurality of disease-specific cross-modal regression models reflects a disease-specific association among different diagnostic modalities: physiological findings in functional data, morphological features in brain imaging data and/or laboratory analysis findings in laboratory data.

In accordance with an embodiment of claim 7, the classification algorithm detects the correct diagnosis from the plurality of diseases by identifying the disease-specific cross-modal regression model that optimally fits the individual case. The classification analyzes the prediction errors to calculate probabilities for the disease from the plurality of diseases.

In accordance with an embodiment of claim 8, the disease-specific cross-modal regression models are trained on the findings of either cognitively normal individuals or patients diagnosed with a disease from the aforementioned plurality of diseases (e.g., mild cognitive impairment, Alzheimer's disease, etc.).

In accordance with an embodiment of claim 9, the list of treatment options for the aforementioned plurality of diseases comprises cognitive treatment, active music therapy, neuroeducation, physical activity, physiotherapy, acupuncture, dietary or nutrition therapy, herbal medicines, immunotherapy, pharmacotherapy, and any other type of cognition-focused interventions and combinations thereof.

Further objects, embodiments and advantages of the present disclosure will become readily apparent to those skilled in the art from the following detailed description of embodiments having reference to the attached figures, the disclosure not being limited to any particular embodiments disclosed.

### BRIEF DESCRIPTION OF THE DRAWINGS

FIGS. 1-9 are referring to the present invention.

FIG. 1 shows a flowchart illustrating a method 100 for diagnostics and finding treatments for mild cognitive impairment, dementias and neurodegenerative diseases.

FIG. 2 depicts a block diagram illustrating a diagnostics and treatment system 200.

FIG. 3 shows a flowchart of the algorithm of data acquisition 300 for method 100.

FIG. 4 shows a flowchart of an embodiment of the system architecture 400 wherein imaging data are used at the input to a cross-modal regression model 202.

FIG. 5 shows a flowchart of an embodiment of the system architecture 500 wherein radiomics data are used at the input to a cross-modal regression model 202.

FIG. 6 shows a flowchart of the algorithm of preprocessing of MRI data 600 used in the invention.

FIG. 7 shows a diagram of getting optimal timing for treatment due to the diagnostic findings and indications for cognition-focused interventions provided by method 700.

FIG. 8 shows a diagram of the architecture 800 of image-based disease-specific cross-modal models.

FIG. 9 shows a diagram of the architecture 900 of radiomics-based disease-specific cross-modal models.

### DETAILED DESCRIPTION OF THE INVENTION

### Definitions

1. Mathematical model is a numerical or quantitative representation of a real-world system. It may reflect different aspects of the system by describing relationships between variables or making predictions about the future. The input data are the predictors which are used to compute the output value. In the classification models, the output is a category, whereas in the regression models, the output is a number. Hence, classification models can be used to compute a class of events, diagnoses or other entities. Meanwhile, the regression models can prognosticate numerical findings, for example, the examination result expressed in numbers.
2. Machine learning models are a type of mathematical models designed to recognize patterns in data or make predictions. According to the type of output data, they also can be categorized into classification and regression machine learning models.
3. Machine learning system is a computer system programmed to create machine learning models. It trains the models to accurately predict a targeted variable from one or many predictors. The models may differ in architecture which comprises data processing, types and number of input and output data, etc. Once trained, the models can be combined in an ensemble model where multiple diverse base models are used to predict an outcome.
4. A system comprises components that can be assembled in modules.
5. Module means distinct assembly of components that can be easily added, removed or replaced in a larger system. Generally, a module is not functional on its own. In computer software, a module is an extension to a main program dedicated to a specific function. For example, a classification module can be designed to categorize cases.
6. Diagnostics is the process of determining which disease or condition explains person's symptoms and signs. Diagnostic data denote information collected and used in the diagnosis of a disease or condition.
7. Diagnostic modality is a general term which denotes a type of diagnostic examination. To leverage the advances of different modalities, physicians use a multi-modal diagnostic approach with a combination of different diagnostic modalities.
8. Cross-modal models are the models that establish an association between at least two different types of diagnostic data, therefore, they can reveal distinct patterns reflected in disease-specific associations between the findings in different diagnostic modalities. When cross-modal models are trained to predict the results in one type of medical examination, diagnostic findings in another modality should serve as predictors. An alternative way to get cross-modal models is to merge different types of diagnostic data at the input to the model trained to identify the diagnosis.
9. Diagnostic modality is a broad term covering a method of acquiring findings and the specific type of diagnostic data (e.g., brain imaging data, laboratory data, functional data and other data types). Functional data are results in any one or a combination of cognitive, psychophysiological, neurophysiological tests and/or any other type of functional examination and assessment.
10. Laboratory data are results of one or more biochemical, hormonal, immunologic, hematologic, genetic, epigenetic analyses, and any other type of biological data obtained from laboratory tests of human samples: blood, cerebrospinal fluid, epithelium, feces, etc.
11. Brain imaging data are any one or a combination of voxel-based morphometry data, surface-based morphometry data, any other type of radiomics findings, brain-imaging data, angiography findings, metabolic imaging data, and blood oxygen level dependent images. Radiomics data is a specific type of imaging data.
12. Radiomics is a mathematical method that extracts a large number of features from diagnostic images using data-characterization algorithms. These features, termed radiomics features, have a potential to uncover tumoral patterns and characteristics that fail to be perceived with the eyes.
10. Computing devices mean machines used to acquire, store, analyze, process, and publish data and other information electronically, including accessories/peripherals for printing, transmitting and receiving or storing information.
11. Dementia is not a specific disease but rather a general term for severe cognitive impairment. Commonly, dementia and other neurodegenerative diseases are diagnosed at late stages due to weaknesses of unimodal diagnostic approaches. Multimodal diagnostics may outperform the unimodal diagnostics. In advanced stages of the disease, neuronal loss depletes resources of the brain, and the interventions cannot provide modest benefits in cognitive functioning. In the patients with the mild-to-moderate level of cognitive impairment, medications and non-pharmacologic treatment may enhance cognitive processes. The available therapeutic options for cognitive disorders are cognitive treatment, active music therapy, neuroeducation, physical activity, physiotherapy, acupuncture, dietary or nutrition therapy, herbal medicines, immunotherapy, pharmacotherapy, and other cognition-focused interventions or a combination of them.
12. Cognitive testing is the most widely used screening tool for neurodegenerative disorders due to the limited availability or low accuracy of alternative diagnostic options. Neuropsychological tests are time-consuming and of questionable reliability since their outcomes are influenced by confounding factors like depression and scholastic education. According to various research or studies, conventional testing can differentiate healthy ageing from cognitive decline with the accuracy ranging from 58 to 90%. An ideal biological marker identifies dementia at a very early stage before degeneration is observed in brain imaging, functional or laboratory data.
13. Multimodal diagnostics is a diagnostic approach combining diagnostic data of at least two diagnostic modalities for making an early and reliable diagnosis. With this approach, more variables can be taken into consideration than are known to specific medical teams or clinics. According to conventional research, it is found that routine brain imaging investigations have their diagnostic and prognostic capacities boosted by functional data obtained at cognitive assessment. In multimodal diagnostics, the benefits of the distinct modalities are leveraged, while their deficiencies are minimized. The examples of multimodal diagnostics comprise any combinations of cognitive scoring with Magnetic Resonance Imaging (MRI) or Positron Emission Tomography (PET).
14. Therapeutic plan is a set of written instructions and records related to the treatment of an illness. Therapeutic plans are documentation tools often considered essential to providing well-rounded health care. The plan may outline specific goals for therapy and interventions, the algorithm of ordering different therapeutic options, their specific indications and contraindications.

The invention relates a method for diagnostics and finding treatments for mild cognitive impairment, dementias and neurodegenerative diseases and a system to incorporate the aforesaid method. The invention thus may lead to advanced interventional strategy and improved outcomes of treating cognitive disorders due to multimodal diagnostics. By relating to brain imaging data, laboratory data, and functional data, e.g., cognitive scores, and methods of data analysis which may be useful in diagnosing and treating cognitive disorders and other diseases known to impair brain function the predictability of illnesses is improved. More specifically, the invention describes methods to detect indications for cognition-focused interventions and finding treatments for the aforementioned pathologies.

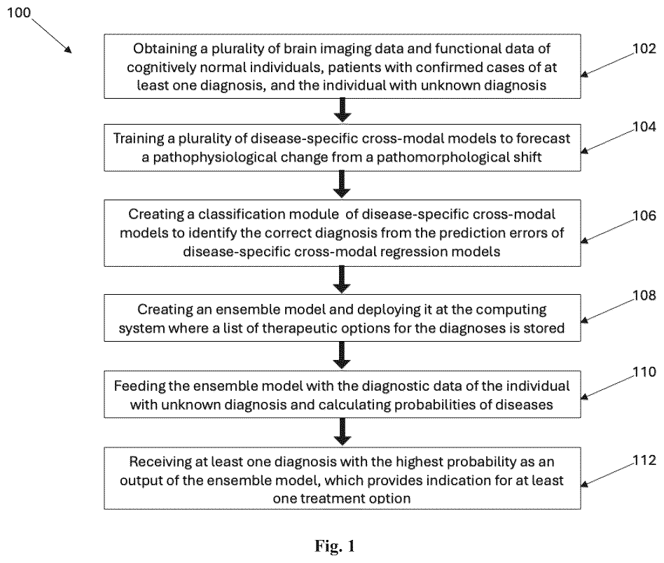

A working prototype of the invention is shown in FIG. 1 in the form of a flowchart illustrating a method 100 for diagnostics and finding treatments for mild cognitive impairment, dementias and neurodegenerative diseases in accordance with an embodiment of the invention. Method 100 incorporates several steps. The first step (102) is the collection of brain imaging data and functional findings of cognitively normal individuals, patients with confirmed cases of at least one diagnosis, and an individual with unknown diagnosis.

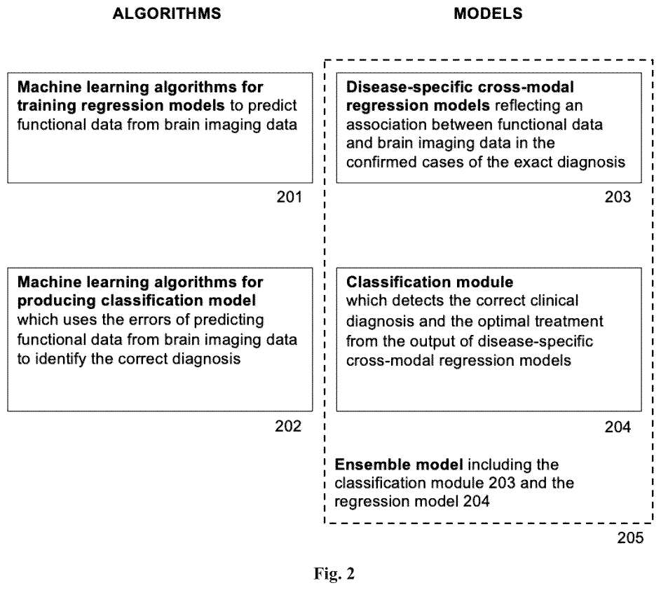

The second step (104) is the construction of disease-specific cross-modal models 203 according to machine learning algorithm 201 depicted in FIG. 2. In the working prototype, regression models 203 are constructed to predict functional performance in cognitive tests from brain radiomics data. In other words, data acquired from voxel—and surface-based brain morphometry are used as predictors of the models trained one by one on the medical findings of the studied groups of patients and healthy adults. The models reflect structure-function association patterns specific for each diagnosis. The pattern serves as a “stamp” of the disease on which the model was trained.

The next step 106 is the production of classification module 206 according to machine learning algorithm 202. In the working prototype, this step is arranged as follows. After training the disease-specific structure-function regression models, the difference between the predicted and actual test scores is calculated. Then, the difference is used to train a classification model which identifies the correct diagnosis. In other words, the machine learning system prepares a classification module based on the prediction errors of the disease-specific cross-modal regression models and the information on the correct diagnoses of the patients.

In step 108, disease specific regression models of step 104 and classification module of step 106 are merged to form an ensemble model 205 visualized in FIG. 2. In this way, FIG. 2 provides a detailed explanation of steps 104-108. Other embodiments of this aspect include corresponding architecture, apparatus, and computer programs recorded on one or more storage devices, each configured to perform the actions of the methods.

In step 110, the examiner enters results of a multi-modal examination of the examinee with unknown diagnosis into a computing device with the ensemble model deployed on it, the base estimators of the ensemble model predict one type of data from at least one other type of data, and the classification module of the ensemble model uses deviations from the disease-specific cross-modal regression models to find the best-fitting model, calculate probabilities for different diagnoses, and output the diagnosis with the highest probability. It can also output the second best treatment-what is helpful if the first treatment is not available.

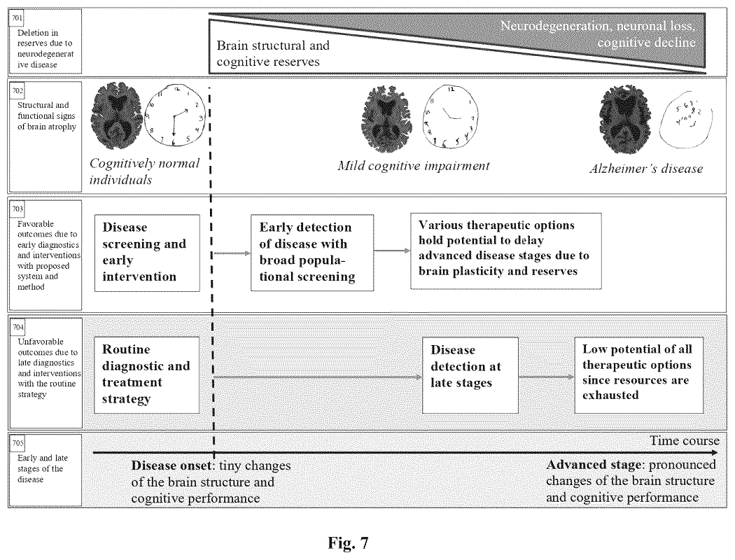

In step 112, the system user receives at least one diagnosis that has the highest probability and selects at least one treatment option which is feasible for this diagnosis. This approach implements early intervention strategy described in element 703 of FIG. 7 which shows the practical implication of early sensitive diagnostics with the invention.

For creating the prototype, data from a publicly available Alzheimer's Disease Neuroimaging Initiative-1 database was used. It includes subjects diagnosed with MCI, early stage Alzheimer's disease, and healthy elderly controls. Results in MRI examination, cognitive and psychophysiological tests were retrieved from the dataset.

In the working prototype, the aforementioned steps are performed with results in the following cognitive and neurophysiological tests: MMSE, ADAS, RAVLT, DSST, and TMT. When the majority voting technique was used to identify the diagnosis, the classification performance improved up to 91.95% true positive rate for healthy participants, 86.21%—for mild cognitive impairment and 80.18%—for dementia cases, which is higher compared to the unimodal diagnostics with cognitive tests.

The performance of the created system is compared for diagnostic accuracy of the invented system with the known methods. The working prototype outperforms existing diagnostic solutions of Tóth, Müller, Fernández-Fleites, and Rashedi: in their solutions the accuracy of tests distinguishing mild cognitive impairment from healthy aging ranges from 58 to 90% [Tóth, L., Hoffmann, I., Gosztolya, G., Vincze, V., Szatlóczki, G., Bánréti, Z., et al. (2018). A speech recognition-based solution for the automatic detection of mild cognitive impairment from spontaneous speech. Curr. Alzheimer Res. 15, 130-138. doi:

10.2174/1567205014666171121114930; Müller, S., Herde, L., Preische, O., Zeller, A., Heymann, P., Robens, S., et al. (2019). Diagnostic value of digital clock drawing test in comparison with cerad neuropsychological battery total score for discrimination of patients in the early course of Alzheimer's disease from healthy individuals. Sci. Rep. 9, 1-10. doi: 10.1038/s41598-019-40010-0; Fernández-Fleites, Z., Jiménez-Puig, E., Broche-Pérez, Y., Morales-Ortiz, S., Luzardo, D. A. R., and Crespo-Rodríguez, L. R. (2021). Evaluation of sensitivity and specificity of the ineco frontal screening and the frontal assessment battery in mild cognitive impairment. Dement. Neuropsychol. 15, 98-104. doi: 10.1590/1980-57642021dn15-010010; Rashedi, V., Foroughan, M., and Chehrehnegar, N. (2021). Psychometric properties of the Persian Montreal cognitive assessment in mild cognitive impairment and Alzheimer disease. Dement. Geriatr. Cogn. Disord. extra 11, 51-57. doi: 10.1159/000514673].

Structural MRI examination detects change in the brain in MCI patients at early stage with 77-78% sensitivity and specificity [Taheri Gorji, H., and Kaabouch, N. (2019). A deep learning approach for diagnosis of mild cognitive impairment based on MRI images. Brain Sci. 9:217. doi: 10.3390/brainsci9090217; Statsenko, Y., Habuza, T., Charykova, I., Gorkom, K., Zaki, N., Almansoori, T., et al. (2021). AI models of age-associated changes in CNS composition identified by MRI. J. Neurol. Sci. 429:118303. doi: 10.1016/j.jns.2021.118303; Uzianbaeva, L., Statsenko, Y., Habuza, T., Gorkom, K., Belghali, M., Charykova, I., et al. (2021). Effects of sex age-related changes in brain morphology. Neuroradiology. 63, 42-43. doi: 10.1016/j.jns.2021.118965].

The accuracy of early diagnostics of Alzaheimer's disease with single photon emission tomography ranged from 70 to 90% in different references [Seto, M., Fukushima, N., Yuasa, T., Nakao, Y., Ichinose, K., Tomita, I., et al. (2021). Reappraisal of the cerebral blood flow measured using 123i-i-iodoamphetamine single-photon emission computed tomography in normal subjects and patients with Alzheimer's disease and dementia with Lewy bodies. Acta Med. Nagasakiens. 64, 91-100. doi: 10.11343/amn.64.91; Wang, S., Qi, Y., Jiang, Y., Chi, X., Huang, K., Ruan, C., et al. (2021). Analysis of brain perfusion single-photon emission tomography images using an easy zscore imaging system for early diagnosis of Alzheimer's disease. J. South. Med. Univ. 41, 1093-1100. doi: 10.12122/j.issn.1673-4254.2021.07.19]. The accuracy of detecting dementia with event-related potentials electroencephalography examination is also around 90% as shown by Prichep, L., John, E., Ferris, S., Rausch, L., Fang, Z., Cancro, R., et al. (2006). Prediction of longitudinal cognitive decline in normal elderly with subjective complaints using electrophysiological imaging. Neurobiol. Aging 27, 471-481. doi: 10.1016/j.neurobiolaging.2005.07.021. However, the method of electroencephalography has the drawbacks to be operator-dependent, sensitive to movement artifacts, and it is uncommon in the routine clinical practice.

FIG. 2 is a block diagram illustrating a core of a mild cognitive impairment, dementia and neurodegenerative disease diagnostic system 200. The system includes a machine learning algorithm 201 for training disease-specific cross-modal regression models 203 (step 104), a machine learning algorithm 202 which serves for producing a classification module 204 (step 106). The latter is combined with the regression models 203 to build an ensemble model 205 for performing a multi-step data analysis. Other embodiments of this aspect include corresponding architecture, apparatus, and computer programs recorded on one or more storage devices, each configured to perform the actions of the methods.

A machine learning algorithm 201 is used to train a plurality of disease-specific cross-modal regression models 203. In a working prototype, the disease-specific cross-modal regression models 203 are trained to predict results in a functional examination from the brain structural data of either cognitively normal individuals or patients diagnosed with a disease from the plurality of diseases. Notably, the invention comprises separate cross-modal association models for each disease and condition.

Another machine learning classification algorithm 202 serves to program classification module 204. The module is designed to identify at least one disease-specific cross-modal regression model which optimally fits the individual case. For doing this, classification module 204 analyzes deviations from the disease-specific cross-modal regression models (i.e., prediction errors of the models). According to the embodiment of the invention, the minimal deviation from the models serves as a biomarker indicating a particular disease or normal aging.

A working prototype of the present invention includes the consequent application of the following diagnostic procedures and computations. First, healthy individuals, the examinee with unknown diagnosis, and patients with confirmed cases of at least one disease from a plurality of diseases pass any or several neuropsychological or cognitive tests from the following plurality of functional examinations: the mini-mental state examination, the Rey auditory verbal learning test, part B of the trail-making test, the digit symbol substitution test, and/or the Alzheimer's disease assessment scale-cognitive subscale. Second, the examinee undergoes brain MRI examination with the 3D T1-weighted image sequence. The acquired images are then processed with an open-source software (e.g., FreeSurfer) to calculate a plurality of brain morphology data, e.g., the voxel-based morphometry data and/or the surface-based morphometry data. The brain radiomics data are expressed in percentage to the total intracranial volume. Third, the relative volumes of various brain areas are used as predictors in the disease-specific cross-modal regression models 203 that are trained to compute cognitive scores and results in psychophysiological tests. In other words, the regression models 203 predict functional performance in tests from brain radiomics. The models 203 are trained on each study cohort separately: on cognitively normal individuals (CN) or on patients with mild cognitive impairment (MCI), or on patients with Alzheimer's disease (AD). Fourth, the actual individual scores and results in functional tests are compared with the values predicted with the morpho-functional regression models 203 for the plurality of diseases (CN, MCI, AD). Fifth, the correct diagnosis of the confirmed cases are the targeted variables, and the prediction errors of the regression models 203 serve as the input data for training machine learning classification algorithm 202. If the examinee passed a single functional examination, the minimal difference between the predicted and actual test scores identifies the specific disease of the observed individual. If several functional examinations were used, the majority voting technique is employed to indicate the diagnosis in the multigroup classification.

The present invention helps in establishing an interrelation between brain morphometry and neuropsychological functioning or cognitive performance. This invention analyses the cognitively normal elderly and patients with neurodegenerative diseases separately and builds the morpho-functional models 203 specific to the normal or accelerated ageing. The ensemble model 205 is used to detect cases of cognitive deterioration. Further, the combined analysis of neuroimaging and cognitive tests enhances the diagnostics of mild cognitive impairment and early-stage dementia.

This method 100 is therefore appropriate for populational screening as well as for developing early management strategies. The proposed approach gains an advantage from the multigroup classification. The system may train models (203, 205) and classification module (204) of the present invention on medical findings of patients with different neurodegenerative diseases and use the proposed diagnostic solution to identify the level of cognitive impairment and discriminate among these diseases.

This invention broadens the clinical applicability of the diagnostic approach compared to the existing methods that are intended mostly for detecting mild cognitive impairment or unspecified dementia. In accordance with another advantageous embodiment of the present invention, the present invention helps in building an artificial intelligence algorithm with improved sensitivity and specificity in detecting cognitive impairment. Many researchers use a multimodal technique to prognosticate the disease course by combining numerous types of diagnostic data. In contrast to them, the present invention employs disease-specific associations between morphology and performance for diagnostic purposes. This is a distinguishing feature of the present invention.

The equipment required for implementing this invention into practice is highly accessible, which results in its high applicability in medicine for screening purposes. Another advantage of the present invention is the non-invasiveness of the major laboratory tests, functional and diagnostic imaging examinations, in particular, cognitive scoring and a routine structural MRI (a 3D T1-weighted scanning sequence). They are easy to perform and do not require any special preparation from the examinee.

The present invention identifies new diagnostic signs based on brain imaging data in combination with functional assessment data. The proposed models of morpho-functional coupling 203 may identify the features which remain unmentioned when physicians use a single diagnostic modality. Hence, the plurality of disease-specific cross-modal regression models 203 helps in identifying mild cognitive impairment and dementia at a very early stage of the disease, before neurodegeneration is observed in brain imaging and neuropathological or cognitive tests.

This solution possibly possesses the key to the next step for diagnosing mild cognitive impairment, Alzheimer's disease, and other dementias.

It should be noted that the invention has been described with reference to particular embodiments and that the invention is not limited to the embodiments described herein. Embodiments are described at least in part herein with reference to flowchart illustrations and/or block diagrams of methods, systems, and computer program products and data structures according to embodiments of the disclosure. It will be understood that each block of the illustrations, and combinations of blocks, can be implemented by computer program instructions. These computer program instructions may be provided to a processor of a general-purpose computer, special purpose computer, or other programmable data processing apparatus to produce a machine such that the instructions, which execute via the processor of the computer or other programmable data processing apparatus, create means for implementing the functions/acts specified in the block or blocks.

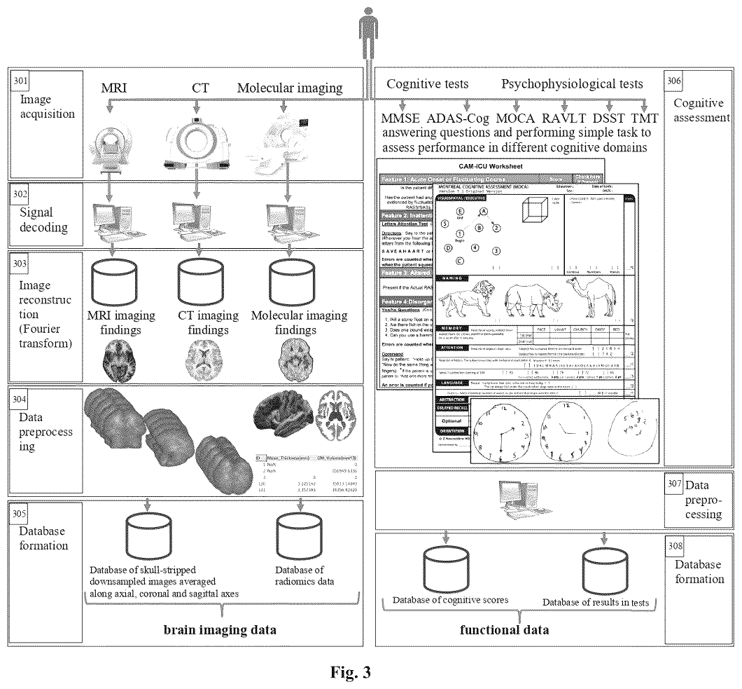

FIG. 3 is a flowchart describing the algorithm of data acquisition 300 for step 102 of method 100 for diagnostics and finding treatments for mild cognitive impairment, dementias and neurodegenerative diseases in accordance with an embodiment of the invention. Morphological findings of the brain are recorded non-invasively with modest methods of radiological examination, e.g., MRI, computed tomography, and positron-emission tomography scanners in step 301. In step 302, raw data obtained from the scanners are decoded, and in step 303, the image is reconstructed according to Fourier transform algorithm. Further, in step 304, algorithms for data preprocessing provide brain imaging data which form the dataset of diagnostic images and radiomics data in step 305. The skull-stripped downsampled images averaged along different planes serve as the input data for image-based disease-specific cross-modal regression models (402). Radiomics findings serve as the input data for radiomics-based disease-specific cross-modal regression models (502).

In accordance with an exemplary embodiment of the present invention, functional findings can be retrieved in the following sequential steps: answering questions in cognitive tests and performing simple tasks in psychophysiological tests in step 306, data preprocessing in step 307, and database formation in step 308. In accordance with another embodiment of the present invention, the data collected in step 308 are used as targeted variables to train image-based and radiomics-based morpho-functional diagnostic models in steps 402 and 502, respectively.

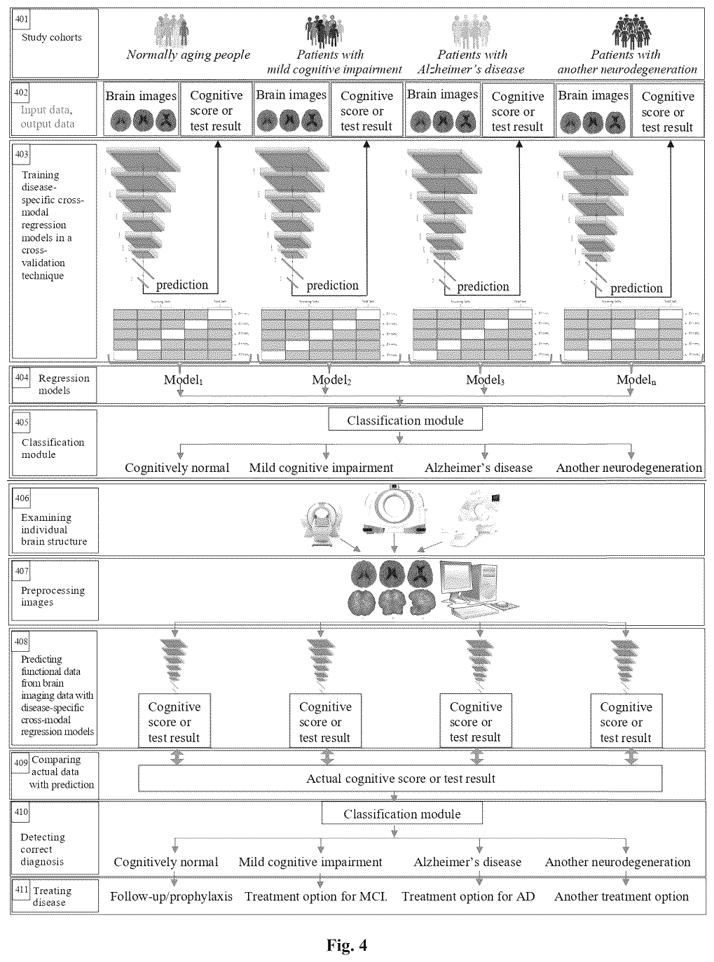

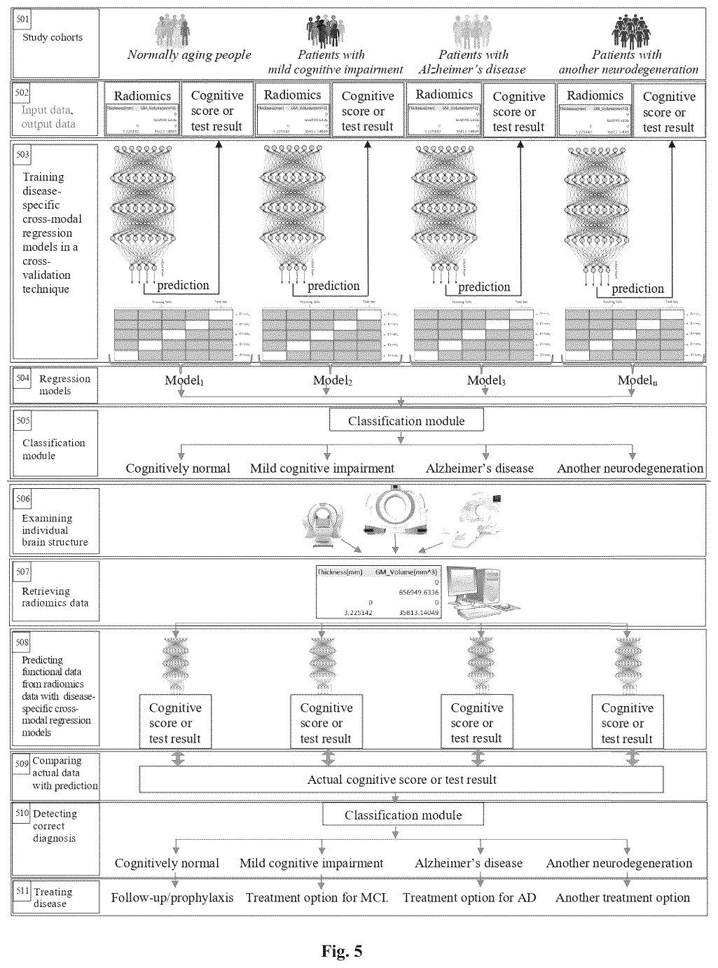

FIG. 4-5 illustrate different architectures of the ensemble model 205 of FIG. 2 and the way how they are created, deployed and used throughout steps 102-112 in FIG. 1.

FIG. 4 is a flowchart showing system architecture 400 for the option in which imaging findings are used at the input to disease-specific cross-modal regression model 202 of the system in accordance with an embodiment of the invention. Steps 401-405 illustrate a way to create the system. First, the examinees are grouped by confirmed diagnosis in step 401. In step 402, brain imaging data serve as predictors, and functional data form the targeted variables for training disease-specific cross-modal regression models in a cross-validation technique in step 403. The designed machine learning models comprise millions of trainable parameters and necessitate high-performance computing resources. In step 404, the regression models predict cognitive scores or test results from brain images. In step 405, the prediction error of the regression models and the correct diagnoses are put into a machine learning system to produce the classification module of the ensemble model. The classification module is programmed to classify cases according to the best fit to at least one disease-specific cross-modal regression model in step 405.

In accordance with an embodiment of the present invention, the application of the system architecture 400 into practice is arranged in a set of steps. First, the examinee undergoes examination of the individual brain structure in step 406. The obtained brain imaging data are preprocessed in step 407, and then they are used to predict the cognitive scores or tests results with disease-specific cross-modal regression models in step 408. The predicted values are compared with the actual cognitive score in step 409, and the classification module identifies the correct diagnosis according to the best fitting diagnostic model in step 410. Once established, the diagnosis serves as an indication for selecting a feasible treatment in step 411.

FIG. 5 is a flowchart describing system architecture 500 for the option in which radiomics data as a special type of brain imaging data are used at the input to disease-specific cross-modal regression models 202 of the system in accordance with an embodiment of the invention. Steps 501-505 illustrate a way to create the system. First, the examinees are grouped by confirmed diagnosis at step 501. In step 502, radiomics data serve as predictors, and functional data are the targeted variables for training disease-specific cross-modal diagnostics models in a cross-validation technique in step 503. Once trained, the regression models predict cognitive scores or tests results from radiomics data in step 504. The deviation of the predicted values from the actual ones are used to create the classification module of the ensemble model in step 505. A machine learning system trains the classification module to classify cases according to the best fit to at least one disease-specific cross-modal regression model in step 505.

The application of the system architecture 500 into practice is arranged in a set of steps. First, the examinee undergoes examination of the individual brain structure in step 506. The obtained imaging findings are preprocessed, and radiomics data are retrieved from the images in step 507. The disease-specific cross-modal regression models predict the cognitive scores or tests results from radiomics data in step 508. The predicted values are compared with the actual cognitive score in step 509, and the classification module detects the correct diagnosis according to the best fitting disease-specific cross-modal regression model in step 510. Once established, the diagnosis serves as an indication for ordering a treatment in step 511.

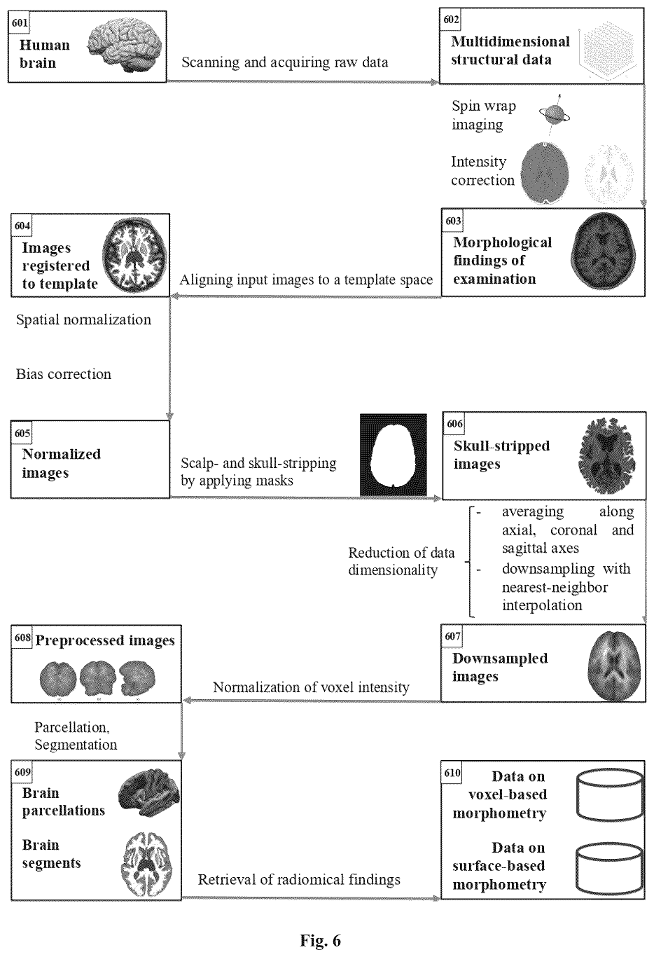

FIG. 6 is a flowchart depicting the algorithm of preprocessing of MRI data 600 used in the method for diagnostics and finding treatments for mild cognitive impairment, dementias and neurodegenerative diseases in accordance with an embodiment of the invention. The algorithms are used to prepare brain images acquired in step 102 for data analysis performed in step 104 of FIG. 1.

First, the examinee undergoes morphological examination of the brain in step 601. The examination provides multidimensional structural data in step 602. Then, all the acquired images are passed through grad-warping and intensity correction frameworks in step 603. In Spin Warp Imaging, phase encoding gradient pulses are applied for a constant duration but with varying amplitude. The Fourier transformation is then used to reconstruct the image from the set of encoded MR signals. Afterwards, the images are registered to a template space in step 604. As human brains differ in size and shape, each brain image is normalized to ensure consistency of orientation by translating the image into a common reference space. In this procedure, the software computes the transformation parameters by using gradient descent at multiple scales to maximize the correlation between the individual volume and an average volume composed of a large number of previously aligned brains. In the subsequent stages, the software will process the transformation matrix which takes image coordinates into the template coordinates.

Neuroimage analysis requires an intensity-homogeneous input. To meet the requirements, the proposed system corrects intensity nonuniformity of an MRI image with algorithms for automatic estimation of the bias fields-smoothly varying low-frequency multiplicative fields caused by coil nonuniformity, magnetic field inhomogeneity, and patient anatomy in step 605. The applied algorithm removes the bias to enhance the quality of segmentation. Otherwise, the classification of voxels into different tissue types is inaccurate due to variations in intensity and contrast across the image. Background removal is used to enhance the predictive value of brain images. Cropping the image to the size of the brain mask allows to remove the background and extract the brain parenchyma with a dedicated software in step 606. Automated stripping of the skull from the image involves deforming a tessellated ellipsoidal template into the shape of the inner surface of the skull.

Then, the voxel intensities are scaled to the standard normal distribution parameters to enhance the predictive performance of the designed machine learning models in step 607.

The proposed method decreases the computational complexity of data analysis by averaging the voxel values along axial, coronal and sagittal axes, which reduces data dimensionality. In this approach, an MRI image is defined as in Formula 1.

$$\\ \begin{matrix}I = \{ ( {v}_{x} , {v}_{y} , {v}_{𝓏} ) : x = \overline{1 , X} , y = \overline{1 , Y} , 𝓏 = \overline{1 , Z} \} & ( 1 )\end{matrix}$$

There, X, Y, Z are the dimensions of the MRI scan in axes x, y and z. The sagittal, coronal or axial slices with the index J can be defined as it is stated in Formula 2.

$$\\ \begin{matrix}{s}_{sagittal}^{{}_{} ( j )} = ( j , {v}_{y} , {v}_{𝓏} ) , {s}_{coronal}^{{}_{} ( j )} = ( {v}_{x} , j , {v}_{𝓏} ) , {s}_{axial}^{{}_{} ( j )} = ( {v}_{x} , {v}_{y} , j ) & ( 2 )\end{matrix}$$

The averaged images are generated according to Formulae 3-5.

$$\\ \begin{matrix}{I}_{sagittal} = \frac{1}{X} {\sum }_{i = 1}^{X} {s}_{sagittal}^{{}_{} ( i )} & ( 3 )\end{matrix}$$

$$\\ \begin{matrix}{I}_{coronal} = \frac{1}{Y} {\sum }_{i = 1}^{Y} {s}_{coronal}^{{}_{} ( i )} & ( 4 )\end{matrix}$$

$$\\ \begin{matrix}{I}_{axial} = \frac{1}{Z} {\sum }_{i = 1}^{Z} {s}_{axial}^{{}_{} ( i )} & ( 5 )\end{matrix}$$

In this way, we average voxel intensities along the sagittal, coronal and axial axes and create two-dimensional datasets Dₐₓᵢₐₗ, D_{sagittal}, and D_{coronal} (see Formulas 6-8).

$$\\ \begin{matrix}{D}_{axial} = \{ {I}_{axial}^{{}_{} 1} , {I}_{axial}^{{}_{} 2} , … , {I}_{axial}^{{}_{} N} \} & ( 6 )\end{matrix}$$

$$\\ \begin{matrix}{D}_{sagittal} = \{ {I}_{sagittal}^{{}_{} 1} , {I}_{sagittal}^{{}_{} 2} , … , {I}_{sagittal}^{{}_{} N} \} & ( 7 )\end{matrix}$$

$$\\ \begin{matrix}{D}_{coronal} = \{ {I}_{coronal}^{{}_{} 1} , {I}_{coronal}^{{}_{} 2} , … , {I}_{coronal}^{{}_{} N} \} & ( 8 )\end{matrix}$$

Subsequently, brain images are down-sampled in the nearest-neighbor interpolation technique in step 608. This allows to match the image size with the input of the machine learning model, i.e. the preprocessed images are used at the input to image-based disease-specific cross-modal regression models in step 402.

Computer software for structural MRI analysis performs parcellation and segmentation in step 609. In voxel-based morphometry, the segmentation process is a multi-step procedure based on intensity information and examination of the segmented regions to label each voxel for reflecting its assignment to a new tissue class. Formulae 9-12 are used to adapt the data to the full skull volume of the individual which is also called total intracranial volume (TIV). To illustrate the way to calculate the absolute and relative volume of brain compartments, we took the following brain parts: cerebrospinal fluid (CSF and CSF %, respectively), intraventricular cerebrospinal fluid (iCSF, iCSF %), grey matter (GM, GM %), and white matter (WM, WM %).

$$\\ \begin{matrix}CSF \% = CSF / TIV & ( 9 )\end{matrix}$$

$$\\ \begin{matrix}iCSF \% = iCSF / TIV & ( 10 )\end{matrix}$$

$$\\ \begin{matrix}GM \% = GM / TIV & ( 11 )\end{matrix}$$

$$\\ \begin{matrix}WM \% = WM / TIV & ( 12 )\end{matrix}$$

In surface-based morphometry, the procedure of cortical parcellation incorporates both geometric information derived from the cortical model, and neuroanatomical convention, as found in the training set. The result is a complete automatic labeling of cortical sulci and gyri based on probabilistic information estimated from a manually labeled training set.

The retrieved voxel-based and surface-based morphometry data are stored in databases in step 610. These databases contain the radiomics data that can be further used at the input to radiomics-based disease-specific cross-modal regression models in step 502.

FIG. 7 is a diagram illustrating practical application 700 of the proposed method 100 in accordance with an embodiment of the invention. The depletion of brain structural and cognitive resources 701 continues across the span of the disease as shown. Element 702 visually presents brain atrophy in brain imaging data and cognitive decline. In accordance with an exemplary embodiment of the present invention, diagnostics and treatment of mild cognitive impairment, Alzheimer's disease or another neurodegenerative disease is more efficient than the conventional approach (703). The proposed method and system allow for high-sensitive populational screening with non-invasive routine diagnostics procedures which are highly available. When the diagnosis is established at early stages, various therapies from the plurality of existing therapeutic options can delay the disease progression since brain reserves are preserved (704). Commonly, the correct diagnosis is established at late stages of the diseases due to low sensitivity of conventional diagnostics procedure. For this reason, treatment is delayed and low-effective in the conventional approach to diagnostics and treatment of mild cognitive impairment, dementia and other neurodegenerative diseases. Element 705 illustrates early and late stages on the time scale of the disease course.

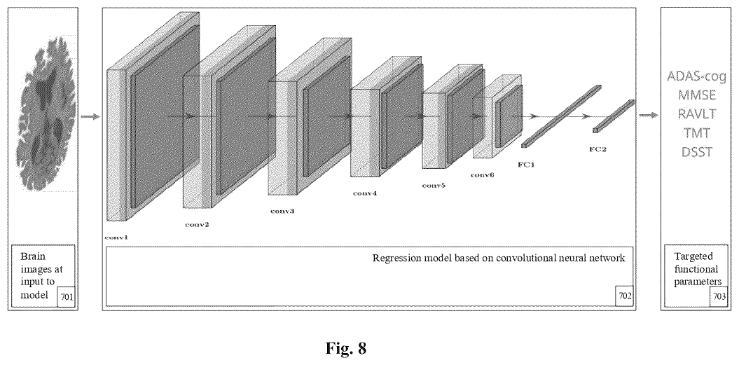

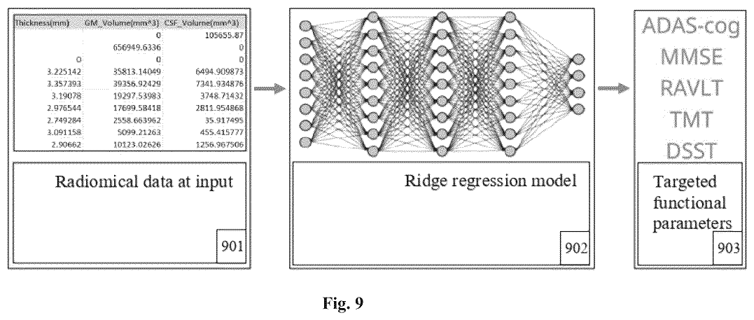

FIG. 8-9 illustrate specific cases of architecture of models 203 trained in step 104.

FIG. 8 is a diagram providing the architecture 800 of image-based disease-specific cross-modal regression models in accordance with an embodiment of the invention. Machine learning is used for building the regression models that predict cognitive scores or results in psychophysiological tests (element 803) from brain images (element 801). The architecture of the model can differ, and the convolutional neural network (element 802) is one of possible solutions among other model architectures.

When used as predictors, image data have high dimensionality, which poses computational challenges. To deal with these challenges, medical professionals need robust computational resources. Deep learning methodologies offer a solution which enables specialists to design and implement regression convolutional neural network models tailored to this purpose. A typical convolutional neural network model has millions of parameters that are trained and optimized through gradient descent techniques. A prototype of the invented system is developed as a model comprising six convolution layers followed by two fully connected dense layers with regularization enforced through an L2 penalty with the alpha hyperparameter of 0.0001. Model training is continued until convergence for a maximum of 200 epochs. Root mean squared propagation is the optimization machine learning algorithm to train the neural network.

At the time of training, validation loss is monitored to tune hyperparameters. The following reduction strategy is implemented in the machine learning algorithm: when the metric plateaus for 10 consecutive epochs, the learning rate should be reduced by 0.2. Time for training is optimized with monitoring validation loss: if it does not reduce in 20 consecutive epochs a termination is triggered. The performance of the model and its generalization ability are assessed in the five-fold cross-validation technique.

The convolutional neural network models are trained on different groups of people separately. These groups are healthy individuals, patients with mild cognitive impairment, Alzheimer's dementia and other neurogenerative diseases. The classification module detects the correct diagnosis with the majority voting technique. Minimal difference between predicted and actual test scores identifies the patient groups.

FIG. 9 is a diagram providing the architecture 900 of radiomics-based disease-specific cross-modal regression models in accordance with an embodiment of the invention. Machine learning is used for building the regression models that predict cognitive scores or results in psychophysiological tests 903 from brain images 901. The architecture of the model can differ, and the ridge regression model 902 is one of possible solutions among other model architectures. The ridge regression model is effective in managing multicollinearity or high dimensionality. Alpha hyperparameter which controls the regularization strength is set to 0.5. To train a reliable model, the objective function's regularization term is multiplied by alpha, which impacts multiple (hundreds) of trainable parameters. During model training, these parameters are iteratively adjusted to minimize the sum of squared errors augmented with a penalty term. These settings ensure a balance between error minimization and parameter control, which allows for building reliable machine learning models.

Ridge regression training has a high computational complexity; this task cannot be performed with mental processes of humans since it requires huge computational power which is available at data science workstations or servers. A multi-fold cross-validation is employed to evaluate model performance across various subsets of data, this technique tests model performance on unseen data to detect overfitting.

The ridge regression models are trained on different groups of people separately. These groups are healthy individuals, patients with mild cognitive impairment, Alzheimer's dementia and other neurogenerative diseases. The classification module detects the correct diagnosis with the majority voting technique. Minimal difference between predicted and actual test scores identifies the patient groups.

It will be apparent to those skilled in the art that various modifications and variations can be made in the present invention without departing from the spirit or scope of the claims. Thus, it is intended that the present invention covers the modifications and variations of this invention provided they come within the scope of the appended claims and their equivalents. The disclosures and the description herein are intended to be illustrative and are not in any sense limiting the invention, defined in scope by the following claims. Many changes, modifications, variations and other uses and applications of the subject invention will become apparent to those skilled in the art after considering this specification and the accompanying drawings, which disclose the preferred embodiments thereof. All such changes, modifications, variations and other uses and applications, which do not depart from the spirit and scope of the invention, are deemed to be covered by the invention, which is to be limited only by the claims.

1. A method with the steps from A to I:
A) Collecting brain imaging data with at least one type of imaging modality from a group of cognitively normal individuals and patients with confirmed cases of at least one diagnosis from a plurality of diseases;
B) Collecting laboratory data with at least one type of laboratory analysis from the group of cognitively normal individuals and patients;
C) Collecting functional data with at least one functional test from the group of cognitively normal individuals and patients;
D) Collecting diagnostics data of the majority of diagnostic procedures of steps A, B, and C from an examinee with unknown diagnosis;
wherein different types of diagnostics data comprise brain imaging data, laboratory data and functional data, and steps A, B, C and D are performed by medical staff;
E) Entering the diagnostic data into a first computing device;
F) Entering at least two types of diagnostic data into a machine learning system which produces cross-modal regression models specific for each diagnosis and gets output values of the models;
G) Entering the output values as predictors and the diagnoses of the group of cognitively normal individuals and patients as targeted variables into the machine learning system which produces a classification module identifying at least one disease-specific cross-modal regression model best-fitting to an individual case;
H) Assembling an ensemble model H from the disease-specific cross-modal regression models and the classification module;
I) Deploying the ensemble model H at the first computing device to calculate probabilities of the diseases, output at least one diagnosis with the highest probability and the optimal therapeutic plan implementing at least one efficient treatment option;
wherein the first computing device may be at least partially different from the machine learning system.

2. A system enabled to run the method according to claim 1, comprising parts:
A) local and/or remote datasets comprising functional and/or laboratory and/or brain imaging data retrieved from diagnostic equipment for imaging of the brain, laboratory analysis of biological samples, and functional tests of human behavior;
B) at least one machine learning system comprising a memory and a processing unit for training a plurality of disease-specific cross-modal regression models to predict at least one type of diagnostic data of part A from at least one other type of diagnostic data of part A;
C) at least one machine learning system comprising a memory and a processing unit for producing a classification module to detect the correct clinical diagnosis and the optimal treatment from the output of disease-specific cross-modal regression models;
D) at least one second computing device comprising a memory for storing a list of therapeutic plans comprising treatment options for each diagnosis and a processing unit for running an ensemble model which incorporates the disease-specific regression models of part B and the classification module of part C to calculate probabilities of mild cognitive impairment, dementias and neurodegenerative disease, output at least one diagnosis with the highest probability and provide indications for a cognition-focused intervention from available treatment options;
wherein the second computing device is used to allocate the machine learning systems of parts B and C and it can work with the datasets of parts A.

3. The method according to claim 1, wherein the brain imaging data are any one or a combination of voxel-based morphometry data, surface-based morphometry data, any other type of radiomics findings, brain-imaging data, angiography findings, metabolic imaging data, and blood oxygen level dependent images.

4. The method according to claim 1, wherein the laboratory data are results in any one or a combination of biochemical, hormonal, immunologic, hematologic analyses, and any other type of biological data obtained from laboratory tests of human samples.

5. The method according to claim 1, wherein the functional data are results in any one or a combination of cognitive, psychophysiological, neurophysiological tests and/or any other type of functional examination and assessment.

6. The method according to claim 1, wherein disease-specific cross-modal regression models predict diagnostic data of one type from the diagnostic data of at least one other type, and each model of the plurality of disease-specific cross-modal regression models reflects a disease-specific association among different diagnostic modalities: physiological findings in functional data, morphological features in brain imaging data and/or laboratory analysis findings in laboratory data.

7. The method according to claim 1, wherein the plurality of diseases is any one or a combination of mild cognitive impairment, mild cognitive impairment, Alzheimer's disease, any type of non-Alzheimer's dementias, a neurodegenerative disease, and other diseases known to impair brain function.

8. The method according to claim 1, wherein the classification module differentiates the correct diagnosis either from healthy status or from a plurality of other diseases and provides indications for the correct treatment of the patient, wherein the classification module is a machine learning classification algorithm which uses the errors of predicting one type of diagnostic data from at least one other type of diagnostic data to calculate probabilities for the diseases from the plurality of diseases.

9. The method according to claim 1, wherein treatment is selected for the disease with the highest probability from the group of treatment options consisting of any one or a combination of cognitive treatment, active music therapy, neuroeducation, physical activity, physiotherapy, acupuncture, dietary or nutrition therapy, herbal medicines, immunotherapy, pharmacotherapy, and any other type of cognition-focused interventions.

10. The system according to claim 2, wherein a computing device stores brain morphology data comprising voxel-based morphometry data, surface-based morphometry data, radiomics findings, brain-imaging data, angiography findings, metabolic imaging data, and blood oxygen level dependent images.

11. The method according to claim 3, wherein the laboratory data stored in the computing device are selected from the group consisting of biochemical, hormonal, immunologic, hematologic analytic data, and any other type of biological data obtained from laboratory tests of human samples.

12. The method according to claim 4, wherein functional examinations are selected from the group comprising cognitive, psychophysiological, neurophysiological tests, any other type of functional examination and assessment and combinations thereof.

13. The method according to claim 5, wherein the disease-specific cross-modal models in the computing device are machine learning regression models trained to predict one type of diagnostic data from at least one other type of diagnostic data of either cognitively normal individuals or patients with confirmed cases of a list of diagnoses.

14. The method according to claim 6, wherein the list of diagnoses comprises mild cognitive impairment, Alzheimer's disease, any type of non-Alzheimer's dementias, a neurodegenerative disease, and other diseases known to impair brain function.

15. The method according to claim 14, wherein the classification module searches for at least one disease-specific cross-modal regression model which optimally fits the individual data of an examinee with unknown diagnosis, calculates probabilities of the diagnoses of claim 14, outputs at least one diagnosis with the highest probability and selects at least one way of treating it from a list of treatment options.

16. The method according to claim 8, wherein the list of treatment options in the computer system comprises any of the following: cognitive treatment, active music therapy, neuroeducation, physical activity, physiotherapy, acupuncture, dietary or nutrition therapy, herbal medicines, immunotherapy, pharmacotherapy, and any other type of cognition-focused interventions, and combinations thereof. The system stores information on the efficiency of treating diseases with various therapeutic options.

17. The method according to claim 1, further comprising the step of implementing the at least one efficient treatment plan on the examince with unknown diagnosis from the optimal therapeutic plan.
